(19) United States
(12) Patent Application Publication
(10) Pub. No.: US 2021/0322025 A1
(43) Pub. Date: Oct. 21, 2021

(54) Temporary Aortic Occlusion Device

(71) Applicants: MicroVention, Inc., Tustin, CA (US); University of Virginia Patent Foundation, Charlottesville, VA (US); United States of America as Represented by the Secretary of the Navy, Silver Spring, MD (US); Emergency Scientific LLC, Salt Lake City, UT (US)

(72) Inventors: George R. Greene, Costa Mesa, CA (US); Ivan Sepetka, Los Altos, CA (US); Cathy Lei, Chino Hills, CA (US); Rupal Nguyen, Costa Mesa, CA (US); Matthew J. Bradley, Silver Spring, MD (US); Stephen T. Ahlers, Silver Spring, MD (US); Carl S. Goforth, Silver Spring, MD (US); James R. Stone, Charlottesville, VA (US); Timothy Ray Nieman, North Salt Lake City, UT (US); Shawn Fojtik, Park City, UT (US)

(73) Assignees: MicroVention, Inc., Tustin, CA (US); University of Virginia Patent Foundation, Charlottesville, VA (US); United States of America as Represented by the Secretary of the Navy, Silver Spring, MD (US); Emergency Scientific LLC, Salt Lake City, UT (US)

(21) Appl. No.: 17/359,304

(22) Filed: Jun. 25, 2021

Related U.S. Application Data
(63) Continuation-in-part of application No. 15/690,152, filed on Aug. 29, 2017.
(60) Provisional application No. 62/382,705, filed on Sep. 1, 2016.
(60) Provisional application No. 63/044,946, filed on Jun. 26, 2020.

(51) Int. Cl. A61B 17/12

(52) U.S. Cl. CPC A61B 17/12109; A61B 17/1204; A61M 25/1011; A61B 2017/1205; A61B 17/12136

(57) ABSTRACT

Blood vessel occlusion devices, systems and methods, in particular embodiments of devices for aortic occlusion. The device includes one or more locator portions and an occluder portion. The device is delivered via transcatheter delivery. The locator portion may be radially expanded using a handle and actuator at the proximal end of the delivery catheter. Tactile feedback from the locator portion is used to determine proper location of the occluder portion, for example within the abdominal aorta. The occluder is then radially expanded to occlude the vessel.

### RELATED APPLICATIONS

This application is a continuation in-part of U.S. application Ser. No. 15/690,152 filed Aug. 29, 2017 which claims priority to U.S. Provisional Application Ser. No. 62/382,705 filed Sep. 1, 2016 entitled Temporary Aortic Occlusion Device, which are hereby incorporated herein by reference in their entirety. This application also claims priority to U.S. Provisional Application Ser. No. 63/044,946 filed Jun. 26, 2020 entitled Temporary Aortic Occlusion Device, which is hereby incorporated herein by reference its entirety.

### FIELD

The present application relates generally to blood vessel occlusion devices, in particular embodiments of devices for aortic occlusion are described.

### BACKGROUND OF THE INVENTION

This application relates to a temporary aortic occlusion device for controlling torso hemorrhage.

Traumatic hemorrhage, primarily the result of blast injuries, is the leading cause of death in active-duty military service members. Although the widespread use of tourniquets has helped to reduce loss of life from severe lower extremity injury, non-compressible torso hemorrhage remains a challenge with high mortality given the relative anatomic inaccessibility of this region to obtain hemorrhage control.

Reports have suggested that up to 25% of hemorrhage sustained in the battlefield is potentially survivable with 50% the result of truncal trauma. Pelvic bleeding, in particular, can be severe and difficult to control, requiring advanced, upper echelon hospital-based care, such as angioembolization, for definitive treatment. Because these advanced care methods and specially trained operators are at higher levels of care, mortality remains high. Unfortunately, outside of pelvic binders for pelvic fracture stabilization, which have limited success in hemorrhage control and are of no value in penetrating trauma there, has been little advancement in the control of non-compressible torso bleeding at the lower echelons of care.

Traditionally, early temporary control for non-compressible torso hemorrhage has been limited to thoracotomy with aortic cross-clamping. This technique has been reserved for moribund patients with absent or lost pulses and has an associated high morbidity and mortality. With growing interest in endovascular techniques for the management of vascular trauma the use of a resuscitative endovascular balloon occlusion of the aorta (REBOA) as an alternative to thoracotomy has been reported. For patients with massive pelvic or intra-abdominal hemorrhage who survive transport to an advanced care facility, placement of a temporary occlusion balloon in the infra-renal aorta, proximal to the aortic bifurcation, or the within the descending thoracic aorta have been used to provide time for more definitive treatment through surgical or endovascular methods. This in-hospital technique provides a method to stop flow of blood below the level of the balloon until the balloon can be deflated under controlled conditions. Insertion of an occlusive balloon is less invasive than a thoracotomy and can be placed in the unstable patient. Endovascular balloon occlusion has been shown to be lifesaving and superior to thoracotomy with aortic cross-clamping in civilian literature.

Placement of a temporary occlusion balloon in the aorta is performed under sterile conditions using ultrasound and fluoroscopic guidance, which requires time, skill, and bulky portable x-ray machines. Despite the potentially life-saving nature of aortic balloon occlusion in the setting of massive torso and/or pelvic hemorrhage, current approaches for the placement these devices require fluoroscopic guidance. Fluoroscopy allows for: (i) intra-arterial injection of contrast dye to define the vascular anatomy, (ii) positioning of an aortic occlusion balloon with respect to this defined anatomy, and (iii) precise control of inflation of the device to allow for sufficient occlusion of the aorta while avoiding over-inflation that could result in rupture of the aorta secondary to balloon inflation.

A technique has been performed utilizing inflation of an aortic occlusion balloon in a trauma bay as a temporary measure for patients with massive pelvic hemorrhage and life-threatening shock, without fluoroscopic guidance. However, this approach requires the expertise of a senior Interventional Radiologist to interpret subtle tactile cues reflecting appropriate balloon placement and inflation. Further, this technique was performed in a “blind” fashion and relied upon the assumption of normal vascular anatomy. In spite of the reported success with balloon occlusion placement, positioning and confirmation has required valuable time, the use of fluoroscopic imaging, and skilled experienced practitioners at higher echelons of care. Additionally, due to the size of the currently available device surgery is required to repair the arteriotomy created by the catheter.

Any non-fluoroscopic approach for temporary occlusion of the aorta in the setting of hemorrhage should address: (i) positioning of the device with respect to individual patient anatomy, (ii) controlled inflation of the balloon or other occlusion device to account for varying aorta diameters, particularly in the under-resuscitated patient, (iii) a low profile, allowing for removal of the device without surgical repair, and (iv) must account for considerations related to the need for operator training in how to safely introduce the device into the femoral artery without creation of additional vascular injury.

Placement of a temporary aortic occlusion device may become an effective technique for hemorrhage control at lower echelons of care if it could be adapted for quicker, easier insertion by non-endovascular specialized providers. For example, Role II facilities such as the Navy Afloat Trauma System (NATS), the Navy/Marine Corps Forward Resuscitative Surgical Systems (FRSS), or Role I settings with Independent Duty Corpsmen and Navy Special Warfare SEAL corpsmen and physicians. Earlier availability of this technique could allow first-responders to stabilize non-compressible torso bleeding until advanced care was available resulting in decreased mortality.

The present invention addresses the need to improve forward surgical applications and targeted therapy for hemorrhagic injury.

### DESCRIPTION OF RELATED ART

Occlusion devices are useful for many applications, for example for controlling torso hemorrhage. Existing solutions for blood vessel occlusion have various drawbacks, including surgical invasiveness and requirements for complex imaging and guidance of the device. A need exists for occlusion devices that overcome these and other drawbacks.

### SUMMARY OF THE INVENTION

Various embodiments of systems, devices and methods for blood vessel occlusion are described. The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for blood vessel occlusion.

The following disclosure describes non-limiting examples of some embodiments. For instance, other embodiments of the disclosed systems and methods may or may not include the features described herein. Moreover, disclosed advantages and benefits can apply only to certain embodiments of the invention and should not be used to limit the disclosure.

The present invention is directed to a temporary aortic occlusion device having an expandable locator portion and an expandable occlusion portion. The expandable locator portion assists a user in determining whether the distal end of the device has been advanced within a patient's aorta, and the occlusion portion is expanded to occlude the patient's aorta, preferably below the renal arteries.

In one embodiment, the locator portion has a maximum expansion diameter that is smaller than a maximum expansion diameter of the occlusion portion. Additionally, the locator portion preferably has a maximum expansion diameter that is the same size or slightly smaller than the internal diameter of a patient's aorta, providing the user with little or no resistance to expansion when positioned in an aorta.

In one embodiment, the locator portion and the occlusion portion are each composed of a plurality of braided mesh wires. Both portions can be coated, laminated, or otherwise covered with a polymer.

In another embodiment, the occlusion portion can include multiple layers of braided wires. These layers can be created from discrete tubular mesh structures or a single, inverted, tubular mesh structure. In another embodiment, the occlusion portion can include an expandable disc structure, woven fabric, and/or spring-biased struts.

In one embodiment, the locator portion is located distal of the occlusion portion. In another embodiment, the locator portion is located proximal of the occlusion portion.

In another embodiment, the occlusion portion is a balloon that can be inflated with a fluid from a proximal end of the device.

In another aspect, an aortic occlusion device is described. The device comprises a delivery catheter extending axially and having a proximal end and a distal end, a first locator, a second locator located proximally of the first locator, an occlusion segment located distally of the first locator and the second locator, and an actuator configured to cause the first locator and the second locator to radially expand.

Various embodiments of the various aspects of the aortic occlusion device may be implemented. The actuator may be configured to cause one or more axially compressive forces to be applied to the first locator and the second locator to thereby cause the first locator and the second locator to radially expand. The actuator may be configured to cause simultaneous radial expansion of the first locator and the second locator. The first locator may be located a first distance from the occlusion segment and the second locator may be located a second distance from the occlusion segment, where the first distance is based on an anatomical distance between a lower-most renal artery and an aortic bifurcation, and the second distance is based on an anatomical distance between a mid-portion of a descending thoracic artery and the aortic bifurcation. The actuator may be configured to cause foreshortening of one or more hypotube segments in a region of the first or second locator, resulting in compression and expansion of the first or second locator.

In another aspect, an aortic occlusion device is described. The device comprises a delivery catheter extending axially and having a proximal end and a distal end, a first locator, a second locator located proximally of the first locator, an occlusion segment located distally of the first locator and the second locator, a first actuator and a second actuator. The first actuator is configured to cause one or more axially compressive forces to be applied to the first locator to thereby cause the first locator to radially expand. The second actuator is configured to cause one or more axially compressive forces to be applied to the second locator to thereby cause the second locator to radially expand.

Various embodiments of the various aspects of the aortic occlusion device may be implemented. The first actuator may be configured to cause foreshortening of one or more hypotube segments in a region of the first locator, resulting in axial compression and radial expansion of the first locator. The second actuator may be configured to cause foreshortening of one or more hypotube segments in a region of the second locator, resulting in axial compression and radial expansion of the second locator. The first locator may be located a first distance from the occlusion segment and the second locator may be located a second distance from the occlusion segment, where the first distance is based on an anatomical distance between a lower-most renal artery and an aortic bifurcation, and the second distance is based on an anatomical distance between a mid-portion of a descending thoracic artery and the aortic bifurcation.

The present invention is also directed to a method of temporarily occluding the aorta of a patient by inserting a temporary aortic occlusion device into a femoral sheath and towards the common iliac bifurcation. An actuation mechanism on the handle of the device is actuated to increase a diameter of a locator on a distal end of the device. If resistance is encountered with the locator, the device is advanced further until the locator can be increased in diameter without resistance. Next, an occluder on the distal end of the device is increased in diameter to occlude the patient's aorta.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a temporary aortic occlusion device according to one embodiment of the present invention utilizing a proximal locator portion and a distal occlusion portion.

FIG. 2 is the temporary aortic occlusion device according to FIG. 1 where the proximal locator portion is in a radially expanded configuration.

FIG. 3 is the temporary aortic occlusion device according to FIG. 1 where both the proximal locator portion and the distal occlusion portion are in radially expanded configurations.

FIG. 4 is the temporary aortic occlusion device according to FIG. 2 in a blood vessel.

FIG. 5 is the temporary aortic occlusion device according to FIG. 3 in a blood vessel.

FIG. 6 is a temporary aortic occlusion device according to another embodiment of the present invention.

FIG. 7 is a temporary aortic occlusion device occlusion portion according to one embodiment of the present invention.

FIG. 8 is a temporary aortic occlusion device occlusion portion according to another embodiment of the present invention.

FIG. 9 is a temporary aortic occlusion device occlusion portion according to another embodiment of the present invention.

FIG. 10 is a temporary aortic occlusion device occlusion portion according to another embodiment of the present invention.

FIG. 11 is a temporary aortic occlusion device occlusion portion according to another embodiment of the present invention.

FIG. 12 is a temporary aortic occlusion device occlusion portion according to another embodiment of the present invention.

FIG. 13 is a temporary aortic occlusion device occlusion portion according to another embodiment of the present invention.

FIG. 14 is a temporary aortic occlusion device handle according to one embodiment of the present invention.

FIG. 15 is a temporary aortic occlusion device handle according to another embodiment of the present invention.

FIG. 16 is a temporary aortic occlusion device according to one embodiment of the present invention utilizing a distal locator portion and a proximal occlusion portion.

FIG. 17 is the temporary aortic occlusion device according to FIG. 16 where both the occlusion portion and the locator portion are in radially expanded configurations.

FIG. 18 is a temporary aortic occlusion device according to one embodiment of the present invention utilizing a proximal balloon and a distal locator portion.

FIG. 19 is the temporary aortic occlusion device according to FIG. 18 where the locator portion is in a radially expanded configuration.

FIG. 20 is the temporary aortic occlusion device according to FIG. 18 in a blood vessel, where both the balloon and locator portions are in radially expanded configurations.

FIG. 21 depicts an embodiment of a temporary occlusion device having an actuator and two locator portions.

FIGS. 22 and 23 depict an embodiment of a distal end of a temporary occlusion device having a locator showing sequential configurations where the locator is axially compressed and foreshortened to cause or allow radial expansion of the locator.

FIG. 24 depicts another embodiment of a temporary occlusion device having two locators and two actuators.

FIG. 25 depicts another embodiment of a temporary occlusion device having a continuous construct for the occlusion and locator portions.

FIGS. 26 and 27 depict another embodiment of a temporary occlusion device having a locator showing sequential configurations of a continuous balloon construct where the locator is expanded prior to the occlusion segment.

FIGS. 28-30 depict another embodiment of a temporary occlusion device in various configurations, the device having mobile constraints that may be controlled by constraint controllers at the handle to allow for specific expansion of either the occlusion or locator segment.

FIG. 31 depicts an embodiment of a temporary occlusion device having a single balloon with variable compliance to form the occlusion and locator portions.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

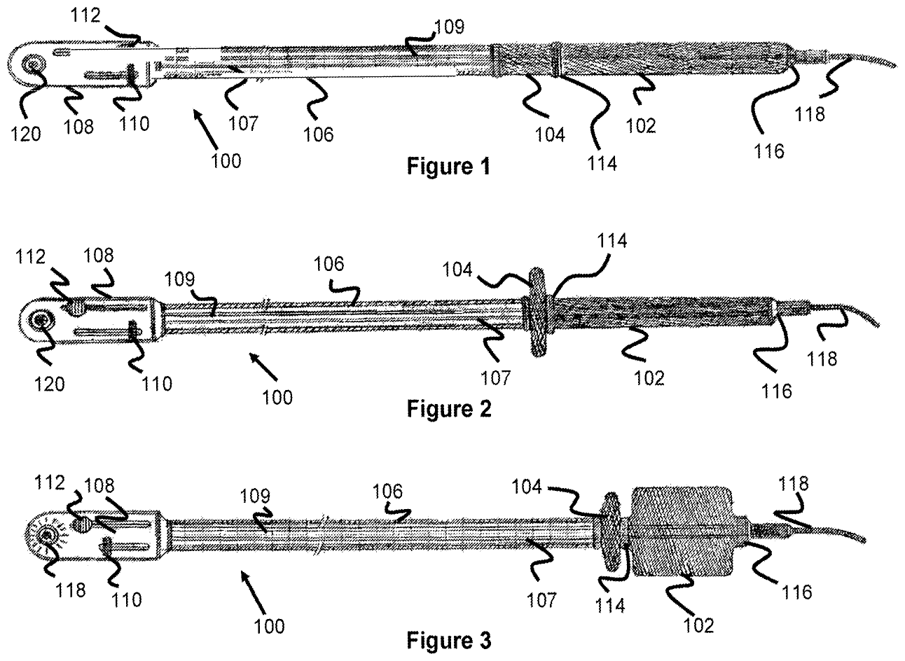

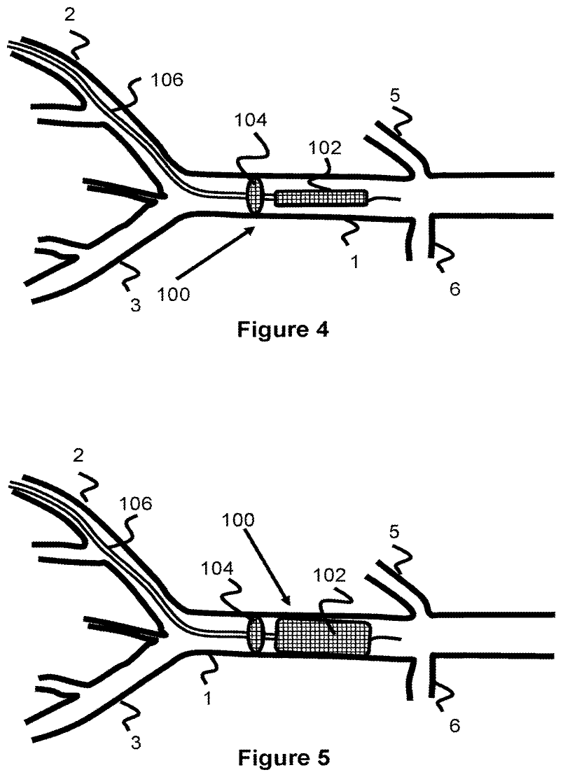

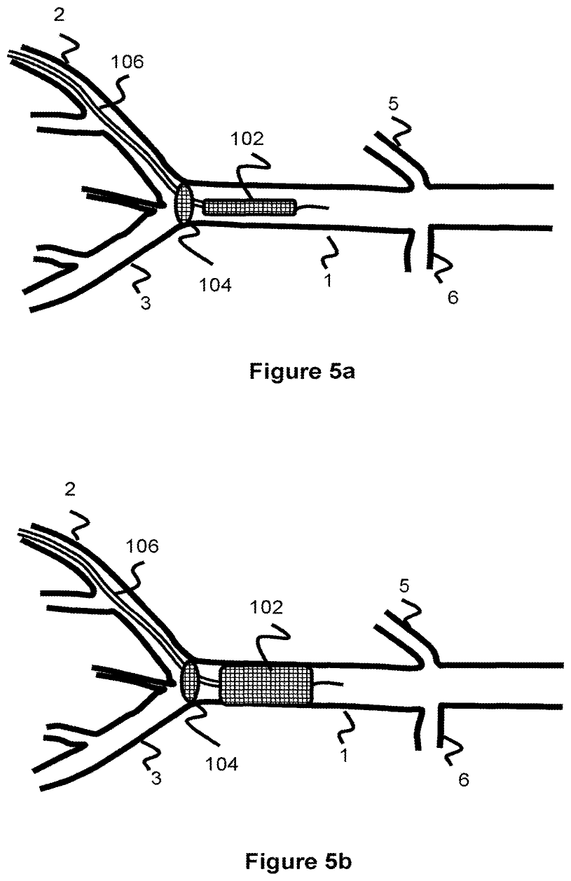

FIGS. 1-5 are directed to a temporary aortic occlusion device 100 that has a radially expandable mesh locator 104 and a radially expandable mesh occlusion portion 102. The device 100 can be loaded in a femoral sheath (e.g., 6F Sheath) and advanced into the common iliac towards the aortic bifurcation target. Once the distal end of the device 100 is close to the target, the mesh locator 104 can be expanded and, if no resistance to the locator 104 occurs, the mesh occlusion portion 102 can be expanded to occlude the aorta.

The locator 104 is preferably composed of a wire mesh (e.g., 0.0005″-0.004″ Nitinol wires) braided into a generally tubular shape. A proximal end of the locator 104 is fixed to distal end of a kink-resistant catheter tube 106 and a distal end of the locator 104 is fixed to ring 114, which is also connected to control wire 109. The control wire 109 is positioned within the lumen of the catheter tube 106 and its proximal end is fixed to slider 112. Hence, as the slider 112 is moved proximally, the control wire 109 moves the ring 114 proximally towards the catheter tube 106, causing the locator 104 to expand. The fully expanded locator 104 can be one of many different sizes, each of which designed to have a maximum expansion that is equal to or smaller than the target aorta size (e.g., 18 mm to 25 mm). The mesh of the locator 104 also may include an elastic hydrophilic coating to prevent blood flow from entering the catheter tube 106.

The occlusion portion 102 functions in a similar manner as the locator 104, having a proximal end fixed to ring 114 and a distal end fixed to ring 116. The ring 116 is further connected to control wire 107, which is slidably positioned within the lumen of the catheter tube 106 and has a proximal end connected to slider 110. Hence, as the slider 110 is moved proximally, it causes the occlusion portion 102 to expand.

The occlusion portion 102 is composed of a wire mesh (e.g., (e.g., 0.0005″-0.004″ Nitinol or PET wires) that are laminated, coated (e.g., dip coating), or have a film applied either on its inner surface, outer surface, or both. Coating materials include polyurethane or silicone, and film materials includes polyethylene, linear low-density polyethylene, polyethylene terephthalate, and Nitinol. In one specific example, each of the wires are first coated in a polymer coating (e.g., polyurethane or polyethylene), braided, and then the inner surface of the occlusion portion 102 is completely coated in a thin 10-15 micron film of the same or similar polymer coating. In another specific example, ePTFE is coated on the inner and outer surface of the occlusion portion 102, “sandwiching” its braid. The occlusion portion 102 optionally has a length greater than that of the locator 104, so as to create a sufficient seal with the patient's aorta.

Preferably, the locator 104 and the occlusion portion 102 are spaced to ensure that the occlusion portion 102 does not occlude the renal arteries leading to the kidneys. A preferred average spacing between the two is about 4.00 cm to about 4.50 cm from each other based on the aortic anatomy of a range of average humans. However, it may be desirable to increase this distance in some circumstances (e.g., large patients) or decrease this distance (e.g., young/small patients).

One aspect of the device 100 is that it allows a user to sense whether there is resistance to expanding the locator 104 or not. In this respect, the locator 104 preferably has a maximum diameter expansion that is either the same size as or slightly smaller than the patient's aorta diameter (e.g., 18 mm to 25 mm). This expansion limit can be limited by the length of movement of the slider 112, as well as the construction of the braid. In contrast, the occlusion portion 102 is configured to have a slightly larger maximum expansion diameter than the locator 104 and/or patient's aorta. This allows the occlusion portion 102 to properly engage the aorta and occlude blood flow. If the device 100 only included the occlusion portion 102 and not the locator 104, a user would encounter expansion resistance prior to entering the aorta, as well as in the aorta, which could cause user-confusion about the device's position. By including the locator 104 that will not substantially encounter resistance in the aorta, the user can have a much higher degree of confidence that the device has entered the aorta.

Since the occlusion portion 102 must be capable of expanding within an aorta 1 and applying a reasonably sufficient force again walls of the aorta 1, there is a risk of rupturing or dissecting the smaller vessels connected to the aorta 1 if expanded too soon. In that regard, the locator 104 can be configured to assist expansion only until encountering a predetermined resistance force and/or with a less forceful expansion force. In this regard, the locator 104 can be expanded with less risk of rupturing the smaller, aortic-adjacent vessels.

One way to achieve this reduced expansion force is to compose the locator 104 of relatively fewer braided wires that, when encountering small amounts of force tend to deform or at least provide less force on the vessels (e.g., 36 0.005″ wires for the locator 104 vs. 48 0.005″ wires for the occlusion portion 102). Additionally, as previously mentioned, the locator 104 can be coated or laminated with a polymer material similar to the occlusion portion 102, which can further create resistance to expansion. Providing a relatively thick coating can further disperse force from the wires of the locator 104, thereby further reducing risk of vessel rupture.

An alternate or additional mechanism includes adding a spring or elastic member between the end of the control wire 109 and the ring 114, such that when resistance is encountered by the locator 104, the spring or elastic expands. Alternately or additionally, the entire control wire 109 can be composed of an elastic material that tends to stretch when resistance is encountered by the locator 104. Optionally, similar mechanisms can be included with regard to the occlusion portion 102, though with the ability to apply somewhat greater force before attenuation.

The handle 108 of the device 100 may also include an indicator light 120 that illuminates when the locator 104 has fully expanded. The handle 108 may have a contact or switch that is triggered when the slider 112 is slid to its proximal-most position to thereby indicate that the aorta 1 has been reached by the device 100.

The distal end of the device 100 also includes an atraumatic tip 118 that is fixed to ring 116. In one example, the tip 118 is composed of a helically-wound wire or coil and is sufficiently flexible to avoid injuring the aorta 1 of a patient.

In operation, the device is loaded directly into a femoral sheath and pushed distally from the femoral artery and into the common iliac towards the common iliac artery bifurcation. Once the catheter tip is close to the target, the slider 112 can be used to slow expand the locator 104. If resistance occurs, the slider 112 can be pushed distally to collapse the locator 104 and the device can be further advanced distally. Once the slider 112 can open fully without resistance, the slider 112 activates the light 120. Finally, the slider 110 can be moved proximally to expand the occlusion portion 102, blocking or occluding the aorta.

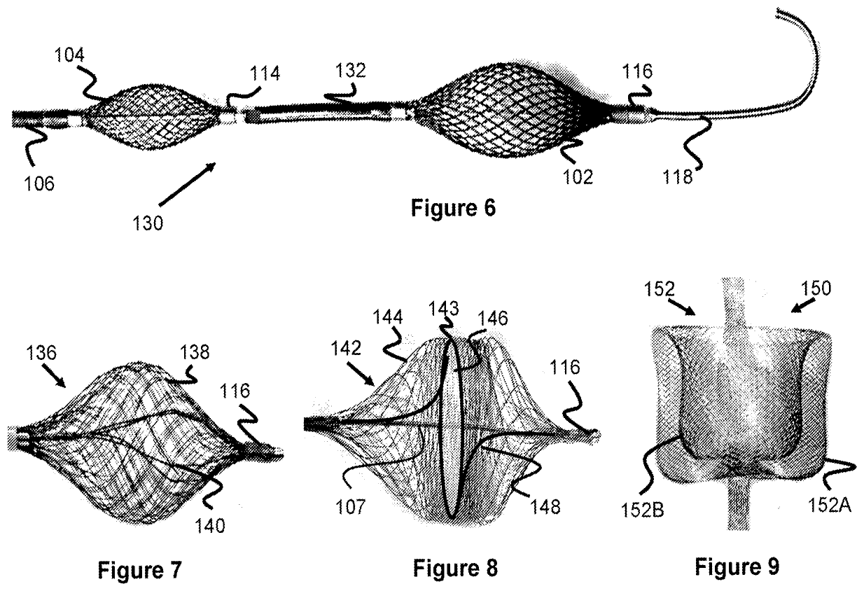

FIG. 6 illustrates another embodiment of an occlusion device 130 that is generally similar to the previously described device 100, however, the locator 104 is spaced apart from the occlusion portion 102 by tubular element 132. This embodiment may be useful if occlusion is desired at a higher location in the patient's aorta.

FIGS. 7-13 illustrate various alternate embodiments of the occlusion portion. For example, FIG. 7 illustrates an occlusion portion 136 braided from a plurality of wires 138 and having a plurality of wire struts 140 disposed within its cavity and connected to the control wire 107. The struts 140 are configured to provide a slight bias or spring-force to help urge the occlusion portion 136 to its expanded configuration. Specifically, the struts 140 can be metal wires connecting between the proximal and distal end of the occlusion portion 136 and that have a shape-memory configuration of a curve (e.g., a curve shape heat set into a shape memory alloy). In a compressed configuration, the struts 140 are relatively straight, but the shape-memory curve of the struts 140 provides an amount of force on the distal end of the occlusion portion 136 to assist the user in its expansion. Alternately, the struts 140 can be configured to return to a relatively straight configuration, biasing the occlusion portion 136 to its compressed configuration. While not shown, the occlusion portion 136 can be laminated, sealed, or otherwise coated in flexible layer of material, as described for other embodiments in this specification.

FIG. 8 illustrates another embodiment of an occlusion portion 142 that is composed of a plurality of braided wires 144. Within the braided wires 144 is a framework comprised of at least a proximal and distal support wires 148 connected to a circular support wire 143. The support wires 148 are connected to each end of the occlusion portion 142 so that, when expanded, the circular support wire 143 is positioned annularly around an axis of the occlusion portion 142. A polymer film 146 is connected to the circular support wire 143, generally forming a plane perpendicular to the axis of the occlusion portion 142. Since the circular support wire 143 and polymer film 146 is sized to expand to substantially the inner diameter of the inner cavity of the occlusion portion 142, an occlusive barrier is created. The braided wires 144 can be left bare or can include a coating, film, lamination, or other occlusive materials as described elsewhere in this specification.

FIG. 9 illustrates another embodiment of an occlusion portion 150 composed of a plurality of braided wires 152 that have a heat-set or memorized shape that causes the wires 152 to form an outer, cup shape 152A and an inner, inverted cup shape 152B. Put another way, the braided wires 152 invert to create two cylindrical layers. The braided wires 152 can be coated, laminated, covered with a film, or used with other occlusive materials as described elsewhere in this specification.

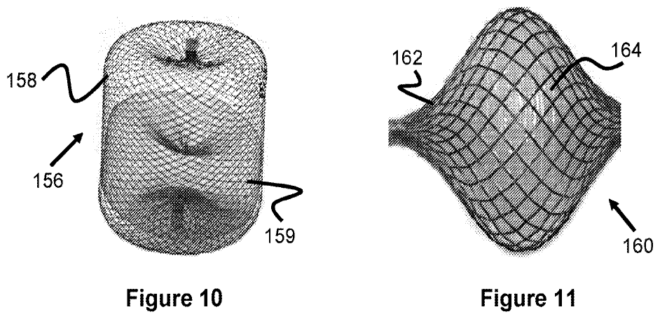

FIG. 10 illustrates yet another embodiment of an occlusion portion 156 having a generally cylindrical outer mesh layer 158 that surrounds an inner, cylindrical mesh layer 159. In one example, the outer mesh layer 158 is composed of relatively larger wires, while the inner layer 159 is composed of relatively smaller wires, which allows the inner layer 159 to have a lower porosity than the outer layer 158, since a greater amount of wires can be used (e.g., a higher pic-per-inch)—this would augment the occlusive effect of the occlusion portion by enhancing the resistance to blood flow once the blood permeates the outer layer. The outer and inner mesh layers 158, 159 can be each formed from a braided, mesh, tubular structure, or can alternately be formed from a single braided, tubular structure that is inverted to form the inner tubular layer 159. Either the outer layer 158, the inner layer 159, or both layers can be coated, laminated, covered with a film, or used with other occlusive material as described elsewhere in this specification.

FIG. 11 illustrates another embodiment of an occlusion portion 160 having a plurality of braided wires forming a mesh layer 162, and an inner layer 164 composed of sealing, hydrophobic material such as polyurethane or silicone layer that is disposed within the mesh layer 162. Optionally, the inner layer 164 can be adhered or physically fastened to the outer mesh layer 162. Optionally, the outer surface of the mesh layer 162 can be coated, laminated, covered with a film, or used with other occlusive material as described elsewhere in this specification.

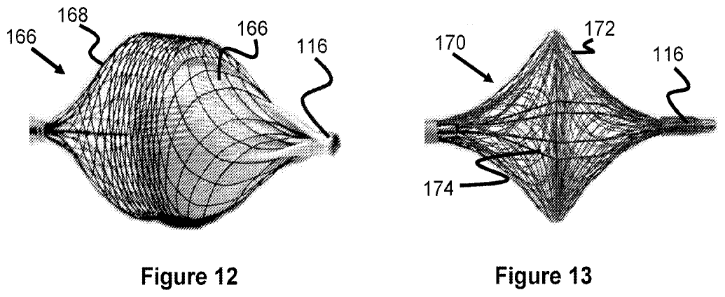

FIG. 12 illustrates another embodiment of an occlusion portion 166 having a plurality of braided wires forming a mesh layer 168, and an inner fabric material 166 fixed at a distal end of the occlusion portion 166. The inner fabric material 166 can be attached to locations around the circumference of the mesh layer 168, or can contain a wire support structure (similar to that formed by the support wires of FIG. 8) that expand the fabric material 166 when the occlusion portion 166 is expanded. The fabric material 166 can in only a proximal or distal half of the mesh layer 168, or can expand within the entire interior of the mesh layer 168. The fabric material 166 can form a funnel shape, a generally spherical shape, or similar shapes, depending on the interior shape of the mesh layer 168. The fabric material 166 can be formed from a woven fabric threads composed of a biocompatible material such as PET. Optionally, the outer surface of the mesh layer 168 can be coated, laminated, covered with a film, or used with other occlusive material as described elsewhere in this specification.

FIG. 13 illustrates another embodiment of an occlusion portion 170 which is generally similar to the embodiment of FIG. 7 in that it has a braided mesh layer 172 that has a plurality of wire struts 174 (e.g., 4) extending between its proximal and distal ends. The struts 174 are bias into a curved shape, such that they provide additional expansion force to the mesh layer 172. The mesh layer 174 forms a generally diamond shape or a shape of two cones connected together. Optionally, the outer surface of the mesh layer 168 can be coated, laminated, covered with a film, or used with other occlusive material as described elsewhere in this specification.

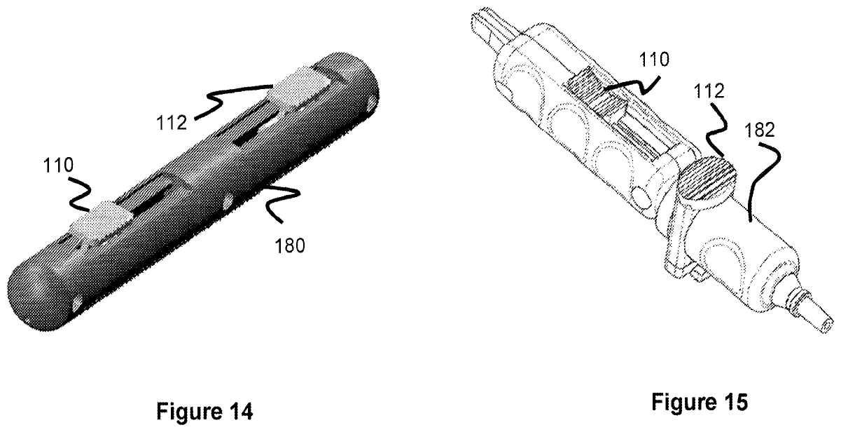

Turning to FIGS. 14 and 15, two different embodiments of handles (180, 182) are illustrated. These embodiments arrange the sliders 110, 112 in line with each other, instead of side-by-side, as in prior embodiments. Additionally, the handle 182 includes a slider 112 that is disposed entirely around the distal portion of the handle 182 and slides in a coaxial manner proximally and distally on the handle 182, the tracks are not shown but in such an embodiment slider 112 would have tracks that it slides on similar to the track that slider 110 slides on. In another embodiment, slider 112 could rotate in order to translate a connected wire—in this embodiment slider 112 would mate over the control wire in a ratcheting-type engagement where rotating slider 112 would translate the control wire which is connected to slider 112.

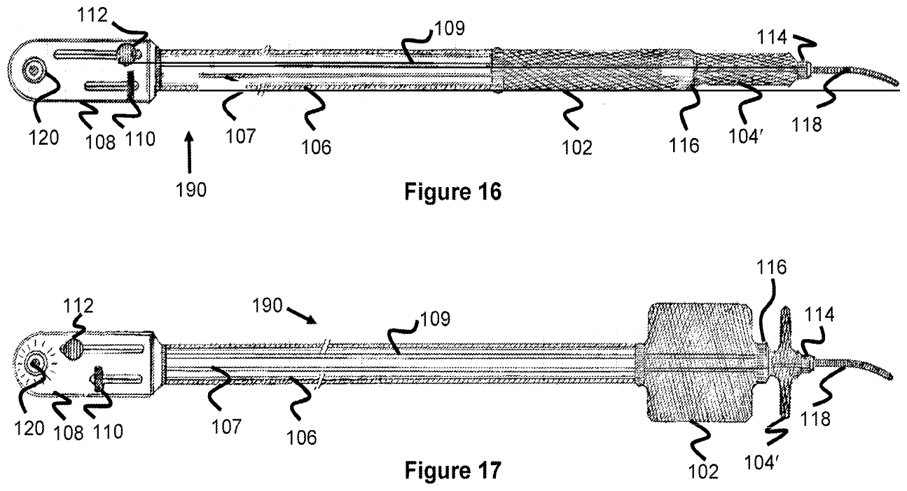

FIGS. 16 and 17 illustrate another embodiment of a temporary occlusion device 190 that is generally similar to the device 100 shown in FIGS. 1-5. However, the locator 104′ is positioned distal of the occlusion portion 102.

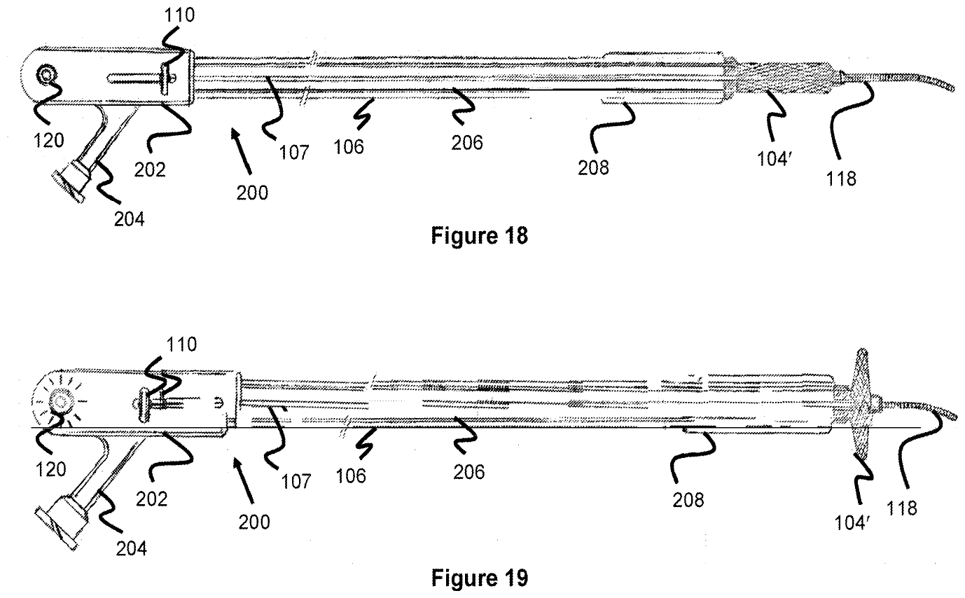

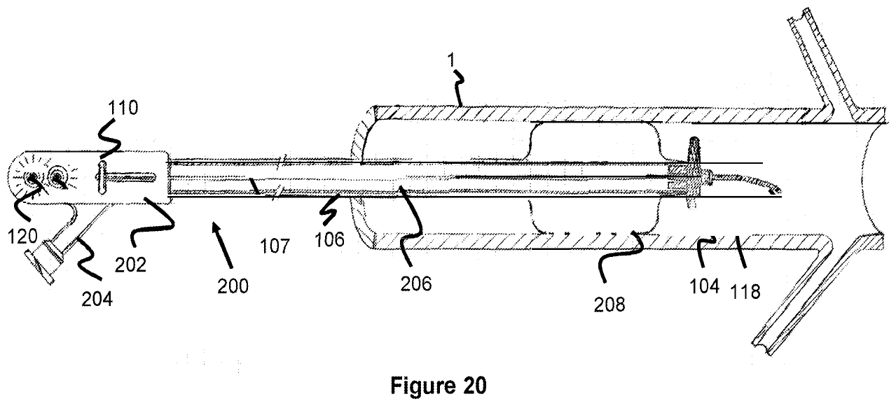

FIGS. 18-20 illustrate yet another embodiment of a temporary occlusion device 200 that is generally similar to the device 190 of FIGS. 16 and 17, including the distal location of the locator 104′. However, instead of a mesh-based occluding portion, a balloon 208 is fixed proximal of the locator 104′ (alternately, the balloon 208 could be fixed distally of the locator 104). A fluid connection port is connected for a fluid source (e.g., a syringe of fluid) and is open to an interior passage 206 within the catheter tube 106, which ultimately connects to an interior of the balloon 208 to allow for selective inflation.

Preferably, the balloon 208 is composed of a highly compliant material. In this respect, if the balloon 208 is over inflated, it will elongate rather than continuing to apply radial force on the wall of the aorta, thereby avoiding balloon-induced aortic damage.

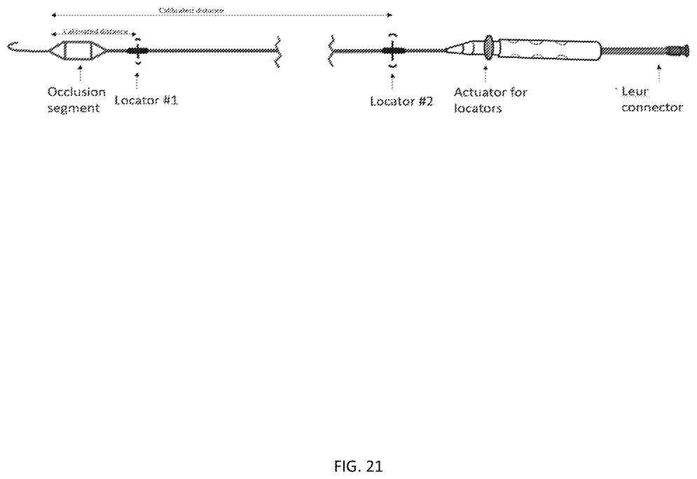

FIG. 21 depicts an embodiment of a temporary occlusion device. This device consists of an expandable occlusion segment and two locators, each with a distance precisely calibrated between the occlusion segment and locator. The occlusion segment can be used to temporarily occlude a vessel, such as an aorta of a patient.

The occlusion segment could be a balloon or wire mesh coated with fluid impermeable material. For balloon embodiments, a leur connector may be used whereby fluid is introduced and flows to the occlusion balloon segment to cause radial expansion of the occlusions segment.

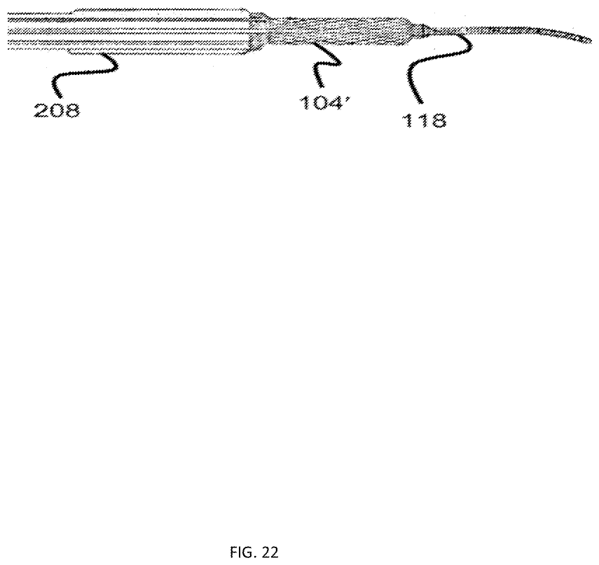

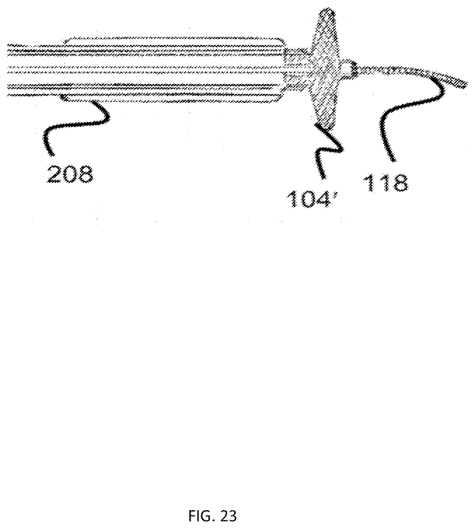

On the handle is a single actuator which deploys both locators #1 and #2 once triggered. Locator deployment is accomplished by foreshortening mechanisms in the region of the locator (for example, foreshortening of one or more hypotube segments in the region of the locator), resulting in compression and expansion of the locators. Examples of foreshortening mechanisms are described herein with respect to FIGS. 22 and 23.

The choice of locator used may be guided by which calibrated distance was desired to be used. The calibrated distances can include distances between two anatomical reference points, for example, a first reference point for desired placement of the occlusion segment and a second reference point for desired placement of the locator when the occlusion segment is at the first reference point. For example, a distance between locator #1 and the occlusion segment could be a precisely calibrated distance between the aorta bifurcation and the lowest renal artery, while a distance between locator #2 and the occlusion segment could be a precisely calibrated distance between the aorta bifurcation and the mid-portion of the descending thoracic artery. Distances can be calibrated based on mean distances and standard deviations across the general human population or measurements taken from a subset thereof.

Based on the desired position of the occlusion segment, one of the locators #1 and #2 can be chosen for determining a relative position of the occlusion segment within the anatomy of a patient. For example, the device would be inserted a certain distance into the anatomy of the patient. The locators #1 and/or #2 can be deployed. After deployment, the device can be retracted so that the locator #1 or locator #2 provides tactile feedback, for example, through frictional resistance relative to an anatomical feature.

In some embodiments, for example, the locator #1 and/or locator #2 can be dimensioned and/or otherwise configured to expand within the aorta and move therein. In some embodiments, the locator #1 and/or locator #2 may have small enough dimensions to move within the aorta when expanded. In some embodiments, the locator #1 and/or locator #2 can have dimensions larger than the aorta when expanded but may be sufficiently compliant to move therein. After expansion of the locator #1 or #2 within the aorta, the locator #1 or #2 can be retracted. The locator #1 or #2 can be dimensioned to provide tactile feedback when withdrawn into contact with the proximal side of the iliac artery or the aorta bifurcation (or other anatomical reference point in other embodiments), for example, in the form of tensional resistance to further withdrawal of the locator #1 or #2. This can be achieved by a locator #1 or #2 dimensioned and/or otherwise configured to resist withdrawal beyond the aortic bifurcation (or other anatomical reference point in other embodiments). For example, in some embodiments, at least one segment of the locator #1 or #2 can have a diameter and stiffness sufficient to prevent passage of the locator #1 or #2 beyond the bifurcation (or other anatomical reference point) when in the deployed state. Tactile feedback can be provided by the locator #1 or #2 providing haptiac feedback when the locator #1 or #2 interacts with a vessel having a correspondingly smaller lumen.

In some embodiments, the device would be inserted a certain distance to utilize the tactile feedback afforded by locator #1 as it interacts with the aorta bifurcation or other anatomical reference point.

In other embodiments, the device would be inserted a certain distance past the location for use of locator #1 to allow for utilization of the calibrated distance for locator #2.

In some embodiments, one or more of the occlusion segment, the locator#1 and/or locator #2 can have a variable stiffness along a radial length thereof. Radial refers to a direction perpendicular to an axial direction of the locator, which axial direction may correspond to an axial direction of a portion of the delivery catheter to which or near which the locator is attached. In some embodiments, the occlusion segment, the locator #1, and/or the locator #2 can have a greater stiffness near a radial center of the occlusion segment, the locator #1, and/or the locator #2 than near the outer radial ends of the occlusion segment, the locator #1, and/or the locator #2. The stiffness at the radial ends of occlusion segment, the locator #1, and/or the locator #2 can be sufficiently low such that the occlusion segment, the locator #1, and/or the locator #2 are atraumatic to the aorta (or other vessel) when expanded and moved therein. The stiffness at the center of the locator #1 and/or #2 can be high enough to facilitate tactile feedback as described herein.

In some embodiments, in which the occlusion segment, the locator #1, and/or the locator #2 is formed of a wire mesh, the stiffness and flexibility can be a result of wire density, spacing of wires, number of braided mesh wires, picks per inch, number of wire turns per inch, braid angle, number of carriers, number of wires in a 360° structure, other suitable structural variations, or combinations thereof. One or more of these variables can be differed at different sections of the occlusion segment, the locator #1, and/or the locator #2 to control the stiffness and flexibility thereof In embodiments in which the occlusion segment, the locator #1, and/or the locator #2 is formed of a balloon, wall thickness of the balloon can be varied, for example by having a thicker wall near a center and a thinner wall near the ends.

FIGS. 22 and 23 depict an embodiment of a distal end of a temporary occlusion device having a locator 104′. The features for radial deployment of the locator described with respect to FIGS. 22 and 23 may be used with any of the occlusion devices described herein.

FIG. 22 depicts the locator 104′ prior to deployment. FIG. 23 depicts the locator 104′ after deployment to its expanded configuration. As shown in FIGS. 22 and 23, the locator 104′ can be compressed axially (i.e., compressed between a proximal end and a distal end of the locator 104′) to cause radial expansion of the locator 104′.

The locator 104′ can be compressed by one or more foreshortening mechanisms. In some embodiments, the proximal end of the locator 104′ can be moved towards the distal end of the locator 104′ while the distal end of the locator 104′ is held in place to cause axial compression and radial expansion of the locator 104′. For example, in some embodiments, a portion of the device coupled to the proximal end of the locator 104′, such as a sheath or deployment lumen (e.g., an inflation catheter, a hypotube, etc.) can be pushed distally while the distal end of the locator 104′ is held in place by another portion of the device such as a sheath, a deployment lumen, or a wire, or is otherwise axially stationary.

In some embodiments, the distal end of the locator 104′ can be moved towards the proximal end of the locator 104′ while the proximal end of the locator 104′ is held in place or is otherwise axially stationary. For example, in some embodiments, a portion of the device coupled to the distal end of the locator 104′, such as a sheath, a deployment lumen or a wire, can be pulled proximally while the proximal end of the locator 104′ is held in place by another portion of the device, such as a sheath or a deployment lumen.

In some embodiments, the occlusion segment can be radially expanded through any of the same or similar mechanisms as the locator, or as otherwise described herein.

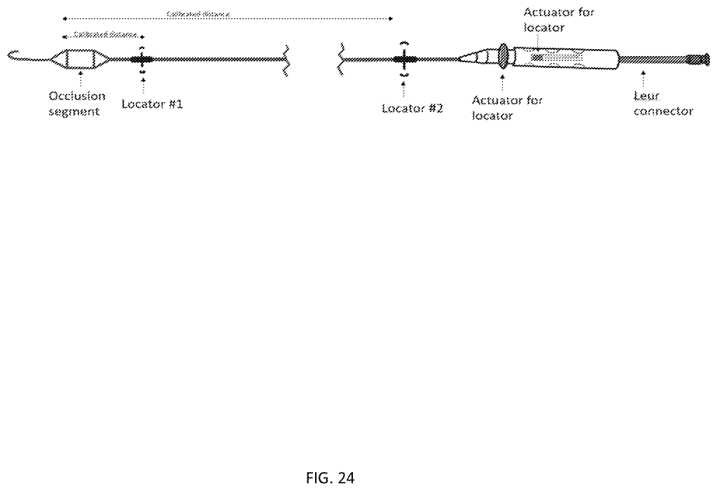

FIG. 24 depicts an embodiment of a temporary occlusion device. The device of FIG. 24 can include any of the same or similar features or functions as the devices of FIGS. 21-23, and vice versa. This device consists of an expandable occlusion segment and two locators, each with a distance precisely calibrated between the occlusion segment and locator. The occlusion segment may be a balloon or wire mesh coated with fluid impermeable material.

In contrast to the embodiment of FIG. 21, which includes a single actuator for both locators #1 and #2, the handle of the embodiment of FIG. 24 includes two separate actuators configured to individually deploy a given locator once triggered. For example, a first actuator can deploy the locator #1 and a second actuator can deploy the locator #2. Locator deployment is accomplished by foreshortening mechanisms in the region of the locator, resulting in compression and expansion of the locators.

The choice of locator used would be guided by which calibrated distance was desired to be used. For example, a distance between locator #1 and the occlusion segment could be a precisely calibrated distance between the aorta bifurcation and the lowest renal artery, while a distance between locator #2 and the occlusion segment could be a precisely calibrated distance between the aorta bifurcation and the mid-portion of the descending thoracic artery.

The device may be inserted a certain distance to utilize the tactile feedback afforded by locator #1 as it interacts with the aorta bifurcation. The device may be inserted a certain distance past the location for use of locator #1 to allow for utilization of the calibrated distance for locator #2.

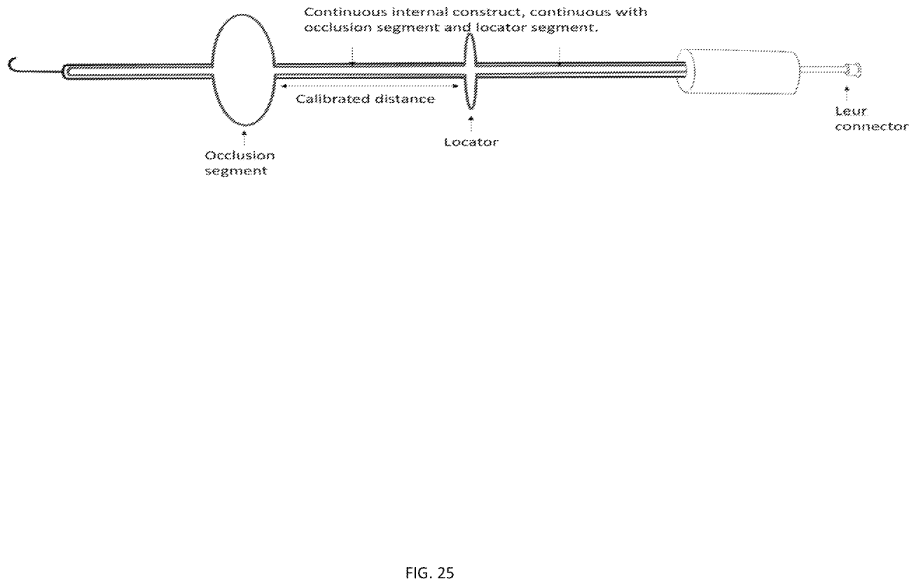

FIG. 25 depicts an embodiment of a temporary occlusion device. The device of FIG. 25 can include any of the same or similar features or functions as the devices of FIGS. 21-24, and vice versa. This device consists of an expandable occlusion segment and a locator with a distance precisely calibrated between the occlusion segment and locator. The occlusion segment and the locator segment are comprised of a single, continuous construct.

The continuous construct is constrained along some segments, but not others, to allow for formation of an occlusion segment and a locator segment that are a calibrated distance from each other when formed. As an example, this construct may be created of balloon material, and inflated at the proximal end of the device by connecting fluid to a leur connector. As another example, the construct may be created of mesh material, which is expanded by actuating a pull wire or wires which result in expansion of these segments.

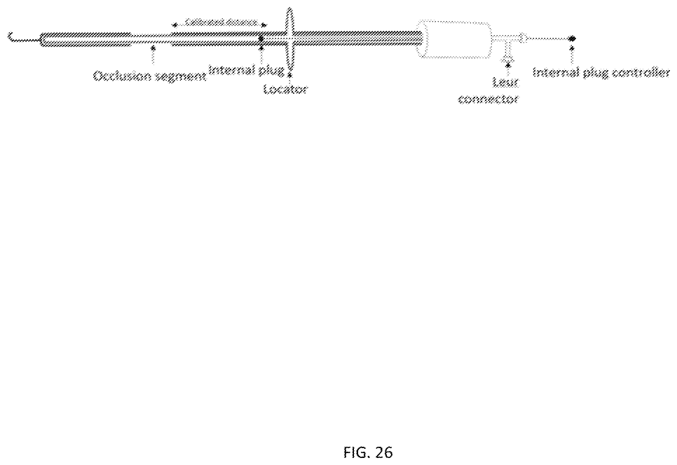

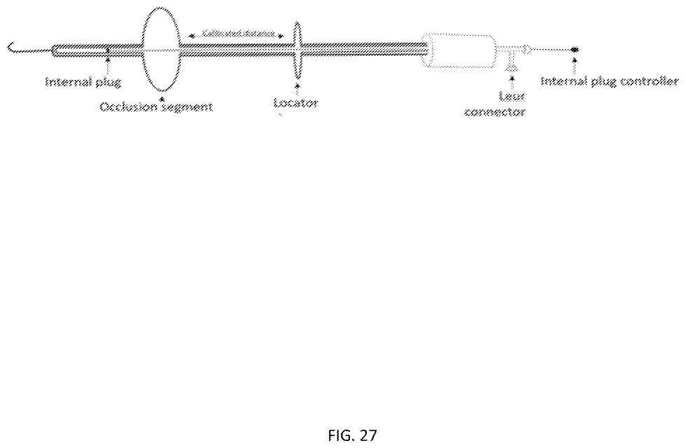

FIGS. 26 and 27 depict an embodiment of a temporary occlusion device. The device of FIGS. 26 and 27 can include any of the same or similar features or functions as the devices of FIGS. 21-25, and vice versa. This device consists of an expandable occlusion segment and a locator with a distance precisely calibrated between the occlusion segment and locator.

The occlusion segment and the locator segment are comprised of a single continuous construct. The continuous construct is constrained along some segments, but not others, to allow for formation of an occlusion segment and a locator segment that are a calibrated distance from each other when formed. As an example, this construct may be created of balloon material, and inflated at the proximal end of the device by connecting fluid to a leur connector.

Expansion of the locator segment separately from the occlusion segment may be accomplished by advancing an internal component with a plug on the distal end. Once the plug is past the locator portion, the locator may be expanded by inflation of the balloon catheter lumen through a sideport connection. Once the plug is advanced past the occlusion segment, both the locator and the occlusion segment may be expanded.

The locator portion may provide tactile feedback when encountering the aorta bifurcation. The occlusion portion would provide occlusion of the aorta.

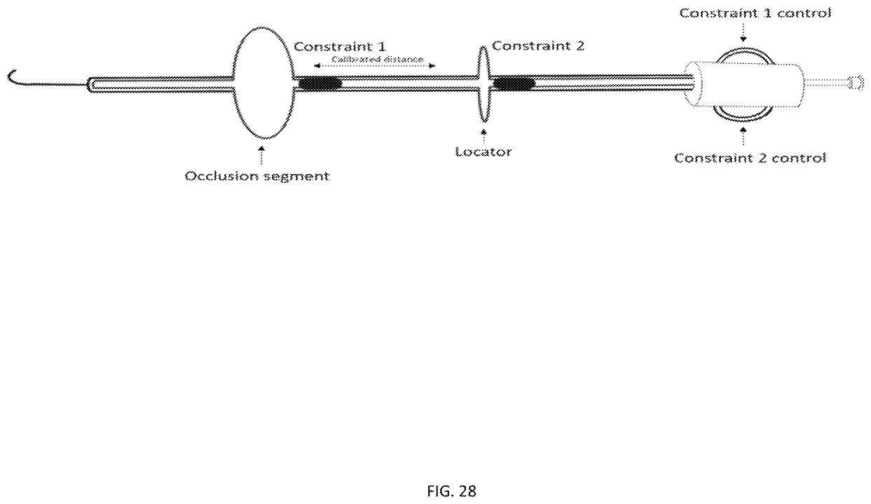

FIG. 28 depicts an embodiment of a temporary occlusion device. The device of FIG. 28 can include any of the same or similar features or functions as the devices of FIGS. 21-27, and vice versa. This device consists of an expandable occlusion segment and a locator with a distance precisely calibrated between the occlusion segment and locator.

The occlusion segment and the locator segment are comprised of a single continuous construct. The continuous construct is constrained along some segments, but not others, to allow for formation of an occlusion segment and a locator segment that are a calibrated distance from each other when formed.

Mobile constraints may be controlled by constrain controllers at the handle to allow for specific expansion of either the occlusion or locator segments. In some embodiments, the constraints may be formed by one or more radial or cylindrical members positioned over the occlusion segment and the locator segment. The constraints may be removed from the occlusion segment and/or locator segment, for example, through retraction, to allow for deployment.

In some embodiments, the constraints may be formed by outer sheath segments, which can be positioned over the occlusion segment and the locator segment to constrain the occlusion segment and the locator segment. The outer sheath segments can be moved away from the occlusion segment and/or the locator segment to allow for deployment. In some embodiments, a single sheath having one or more openings can be used to cover the occlusion segment and the locator segment. The sheath can be moved so that the one or more openings are positioned over the occlusion segment and/or locator segment to allow for deployment. In some embodiments, multiple separate sheath segments are used.

In some embodiments, the constraints can be moved using a pulley mechanism or wire to sheath and un-sheath the occlusion segment and/or locator segment. In some embodiments, the sheath segments position over the occlusion segment and the locator segment can be independently controlled.

The locator portion may provide tactile feedback when encountering the aorta bifurcation. The occlusion portion would provide occlusion of the aorta.

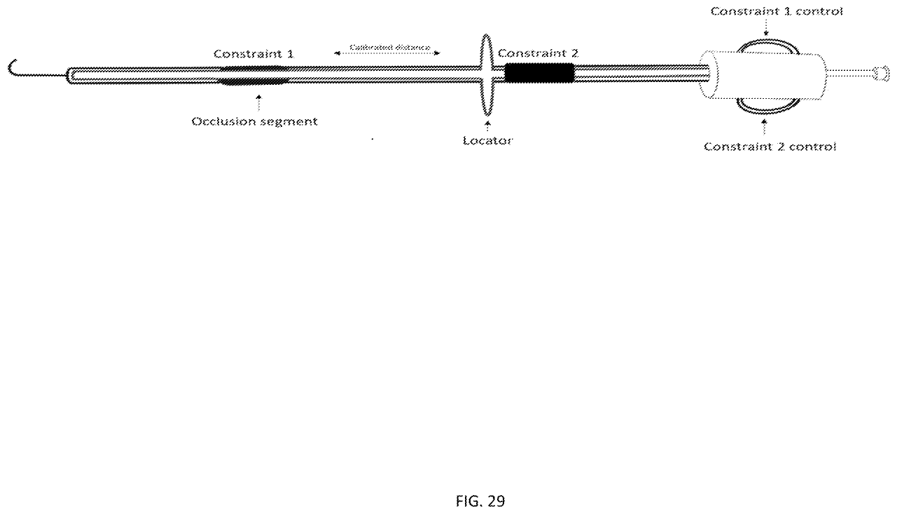

FIG. 29 depicts an embodiment of a temporary occlusion device. The device of FIG. 29 can include any of the same or similar features or functions as the devices of FIGS. 21-28, and vice versa. This device consists of an expandable occlusion segment and a locator with a distance precisely calibrated between the occlusion segment and locator.

The occlusion segment and the locator segment are comprised of a single continuous construct. The continuous construct is constrained along some segments, but not others, to allow for formation of an occlusion segment and a locator segment that are a calibrated distance from each other when formed.

Mobile constraints, for example as described with respect to FIG. 28, may be controlled by constraint controllers at the handle to allow for specific expansion of either the occlusion or locator segments. Shown here is constraint of the occlusion segment.

The locator portion would provide tactile feedback when encountering the aorta bifurcation. The occlusion portion would provide occlusion of the aorta.

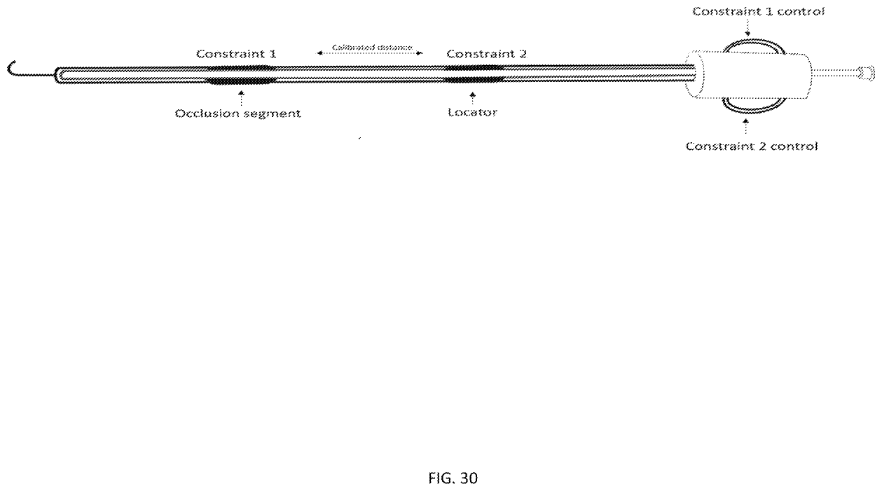

FIG. 30 depicts an embodiment of a temporary occlusion device. The device of FIG. 30 can include any of the same or similar features or functions as the devices of FIGS. 21-29, and vice versa. This device consists of an expandable occlusion segment and a locator with a distance precisely calibrated between the occlusion segment and locator.

The occlusion segment and the locator segments are comprised of a single continuous construct. The continuous construct is constrained along some segments, but not others, to allow for formation of an occlusion segment and a locator segment that are a calibrated distance from each other when formed.

Mobile constraints, for example as described with respect to FIG. 28, may be controlled by constrain controllers at the handle to allow for specific expansion of either the occlusion or locator segments. Shown here is constraint of the occlusion and locator segments.

The locator portion would provide tactile feedback when encountering the aorta bifurcation. The occlusion portion would provide occlusion of the aorta.

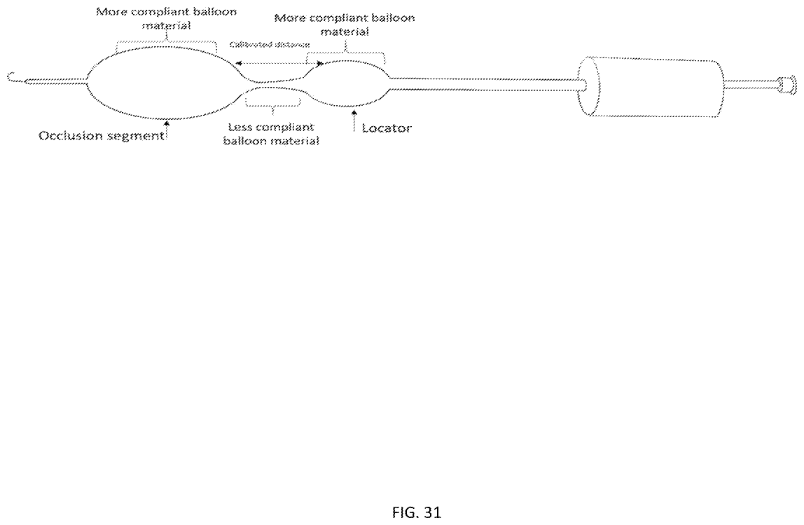

FIG. 31 depicts an embodiment of a temporary occlusion device. The device of FIG. 31 can include any of the same or similar features or functions as the devices of FIGS. 21-30, and vice versa. This device consists of an expandable occlusion segment and a locator with a distance precisely calibrated between the occlusion segment and locator.

The occlusion segment and the locator segment are comprised of a single continuous balloon. This single balloon may be more compliant in select locations of the balloon material, allowing for selected expansion at those locations and not at less compliant locations. The variable balloon compliance may be achieved with variable wall thicknesses, variable layers of balloon material, variable types of material, variable material densities, variable structures adjacent the balloon to impose more or less compliance, other suitable features, or combinations thereof.

More compliant and less compliant sections are calibrated in distance from each other to allow for the more proximal section to serve as an internal anatomic location reference, with the more distal portion serving a vessel occlusion function.

In some embodiments, the locator portion can have different compliance than the occlusion portion, for example, to allow the locator portion to expand before the occlusion portion and/or allow the locator portion to expand to a different size than the occlusion portion. In some embodiments, the locator portion can have a different wall thickness than the occlusion portion and/or allow the locator portion to expand to a different size than the occlusion portion.

The locator portion would provide tactile feedback when encountering the aorta bifurcation. The occlusion portion would provide occlusion of the aorta.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations may be depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An aortic occlusion device, comprising:
a delivery catheter extending axially and having a proximal end and a distal end;
a first locator;
a second locator located proximally of the first locator;
an occlusion segment located distally of the first locator and the second locator; and
an actuator configured to cause the first locator and the second locator to radially expand.

2. The aortic occlusion device of claim 1, wherein the actuator is configured to cause one or more axially compressive forces to be applied to the first locator and the second locator to thereby cause the first locator and the second locator to radially expand.

3. The aortic occlusion device of claim 1, wherein the actuator is configured to cause simultaneous radial expansion of the first locator and the second locator.

4. The aortic occlusion device of claim 1, wherein the first locator is located a first distance from the occlusion segment and the second locator is located a second distance from the occlusion segment, wherein the first distance is based on an anatomical distance between a lower-most renal artery and an aortic bifurcation, and the second distance is based on an anatomical distance between a mid-portion of a descending thoracic artery and the aortic bifurcation.

5. The aortic occlusion device of claim 1, wherein the actuator is configured to cause foreshortening of one or more hypotube segments in a region of the first or second locator, resulting in compression and expansion of the first or second locator.

6. An aortic occlusion device, comprising:
a delivery catheter extending axially and having a proximal end and a distal end;
a first locator;
a second locator located proximally of the first locator;
an occlusion segment located distally of the first locator and the second locator;
a first actuator configured to cause one or more axially compressive forces to be applied to the first locator to thereby cause the first locator to radially expand; and
a second actuator configured to cause one or more axially compressive forces to be applied to the second locator to thereby cause the second locator to radially expand.

7. The aortic occlusion device of claim 6, wherein the first actuator is configured to cause foreshortening of one or more hypotube segments in a region of the first locator, resulting in axial compression and radial expansion of the first locator.

8. The aortic occlusion device of claim 6, wherein the second actuator is configured to cause foreshortening of one or more hypotube segments in a region of the second locator, resulting in axial compression and radial expansion of the second locator.

9. The aortic occlusion device of claim 6, wherein the first locator is located a first distance from the occlusion segment and the second locator is located a second distance from the occlusion segment, wherein the first distance is based on an anatomical distance between a lower-most renal artery and an aortic bifurcation, and the second distance is based on an anatomical distance between a mid-portion of a descending thoracic artery and the aortic bifurcation.
